(19) United States
(12) Patent Application Publication
(10) Pub. No.: US 2023/0337686 A1
(43) Pub. Date: Oct. 26, 2023

(54) METHOD FOR PRESERVING COOKED BAKERY PRODUCTS

(71) Applicant: LESAFFRE ET COMPAGNIE, Paris (FR)

(72) Inventors: Emilie BRYCKAERT, WAMBRECHIES (FR); Florence DELCHAMBRE, QUESNOY-SUR-DEULE (FR); Laurent SOUPIRON, DON (FR)

(73) Assignee: LESAFFRE ET COMPAGNIE, Paris (FR)

(21) Appl. No.: 17/778,171

(22) PCT Filed: Nov. 26, 2020
(86) PCT No.: PCT/FR2020/052178

§ 371 Date: May 19, 2022

(30) Foreign Application Priority Data
Nov. 26, 2019 (FR) FR1913214

(51) Int. Cl. A21D 10/00; A21D 8/04; A23L 3/3571

(52) U.S. Cl. CPC A21D 10/005; A21D 8/045; A23L 3/3571

(57) ABSTRACT

A method for preparing cooked, mold-resistant bakery product, including cooking a dough obtained by a sponge and dough type baking method using a poolish containing from 20 to 55 g/L of ethanol, preferably from 35 to 50 g/L of ethanol, and including, after cooking, a step of applying a solution to the surface of the cooked bakery product that includes a live leaven and/or a live yeast.

The invention relates to a method for preparing cooked bakery products allowing long-term storage without appearance of molds.

The aim of the invention is to offer professionals and consumers mold-resistant cooked bakery products that can be stored for several weeks without deterioration of their appearance, taste and flavor and without addition of non-natural products.

Breadmaking products or cooked bakery products, especially if they have little or no sugar content and if they are sliced and sold in slices, are susceptible to the development of molds during storage. The molds appear after a few days of storage.

Molds are due to contaminants that are always present in the atmosphere of a bakery, which are deposited on the slices of bread. Certain molds produce toxins whose consumption is detrimental to health. Consequently it is often essential to add antimold or antifungal agents such as acetic acid or salts thereof, propionic acid or salts thereof, and sorbic acid or salts thereof, to the composition of breads, and more particularly sliced breads for long-term storage, during their production. Benzoic acid and salts thereof, esters of p-hydroxybenzoic acid or of salts thereof, also called PHB or parabens, are other preservatives used conventionally in the field of food products. In general, mold inhibitors are weak acids and/or salts of weak acids, and the expression “mold inhibitor” denotes all of these products, including all weak acids, also used as acidifying agents.

These antifungal or antimold agents are non-natural preservatives. Nowadays, more and more consumers are looking for products that are free from preservatives. The “clean label” trend is very strong and non-natural preservatives must be avoided.

Alcohol may also be sprayed onto cooked products or injected into cooked products on account of its antifungal properties. However, the products resulting from this application of alcohol are unsuitable for children and for a proportion of the population who cannot absorb alcohol.

These antifungals have a variable inhibitory action on baker's yeasts. In practice, it is calcium propionate that is the most used in breadmaking. It has a moderate but definite inhibitory action on baker's yeasts. Its use leads to longer proof times and to breads having a more-open crumb.

The bread called “pain de mie” in France or “sandwich bread” in the United States, containing fat and sugar, and sliced, is intended for long-term storage, and for this purpose it is necessary to add antimold agents. The same applies to brioches, milkbreads, buns or other breadmaking products for long-term storage.

In general, sandwich bread or buns are prepared by an indirect breadmaking method.

The indirect breadmaking method is a method that comprises a first prefermentation step using part of the flour, water and yeast from the complete recipe. In French bakery terminology this first prefermentation step is called a leaven yeast or a poolish or a sponge, depending on the consistency of the dough. These indirect methods with prefermentation with baker's yeast are little used in France but are the main methods used in the United States of America (USA). The step corresponding to formation of a leaven yeast, which is a pre-ferment in the form of a pasty mass in alcoholic fermentation by yeast, is better known by the American term sponge.

Sponge denotes the first prefermentation step with in general at least 40% of the flour used in the complete recipe, and most of the yeast and water used in the complete recipe. The term dough, which signifies “pâte” in French, corresponds to all the steps in breadmaking (the final step leading to baking of the bread).

The advantage of the prefermentation step is that it allows the yeast to produce a set of metabolites that will improve the taste and the texture of the bread obtained. This generally becomes more pronounced as the sponge contains a higher proportion of the flour from the complete recipe.

As this prefermentation step is intended for maximum expression of the fermentation potential of the yeast and as it is assumed that during the first 45 minutes of leavening, the yeast is very sensitive to its environment (page 629, volume II, Baking Science and Technology, E. J. Pyler; see also standard formula on page 591), the general rule is to add the mold inhibitor or inhibitors during the second step of breadmaking, namely the dough step. This second and last step comprises the conventional breadmaking steps, namely in particular kneading with all the ingredients (such as flour, water, salt, yeast, poolish etc.), dividing the dough, shaping and molding, final proving or proof-time in ovens and baking. Thus, in general, the mold inhibitor or inhibitors are added in the step defined above as the dough step.

The problem with using mold inhibitors in bread and other yeast raised products is that they inhibit the yeast cells at the same time as the unwanted molds. This results in a reduction in activity of the yeast and the production of gas.

Various methods have been used to overcome these problems. As mentioned above, the commonest is always to add the inhibitor in the dough step and never during the step of sponge or liquid starter culture, as it is during this first step that the inhibitor has the most opportunity to have a detrimental action. However, this method has also shown its limits.

The use of these non-natural inhibitors is unsuitable for the current “clean label” trend. Moreover, the use of alcohol, even if it proves effective in its antimold activity, is not suitable for the entire population. Vinegar is one of the only “clean label” alternatives, but is less effective.

Among the other means of combating molds, we may mention those described in WO 2009/097333 and in US 2003/0161911.

The method of preservation proposed by WO 2009/097333 consists of applying a composition comprising live yeast on the products to be preserved. Application may be carried out by any means just before packaging the products to be protected. However, with this method preservation is provided for 21 days at most.

US 2003/0161911 describes yeast strains intended for breadmaking from doughs comprising at least 5% of sugars. It also describes the corresponding breadmaking methods that allow bread to be obtained having a level of ethanol less than 1.5% (w/w) that gives sufficient resistance to the development of molds.

The solutions described above are not entirely satisfactory insofar as they do not meet the needs of the market in terms of storage time of bread, in particular sliced loaves, and may make the baker's work more complicated. Professionals and consumers are becoming more and more demanding and require the production of sliced loaves that remain in a good state of preservation for several weeks while keeping all their properties and organoleptic qualities and without adding non-natural products.

After numerous experiments, the inventors found that the appearance of molds on a sliced loaf, such as French sandwich bread or else American bread, is slowed considerably when said bread is prepared by a method comprising cooking a dough obtained by a breadmaking method of the sponge and dough type and when the bread is treated by the surface application of a composition comprising a live leaven and/or a live yeast.

### SUMMARY OF THE INVENTION

Thus, a method is proposed for preparing mold-resistant cooked bakery products comprising cooking a dough obtained by a breadmaking method of the sponge and dough type using a poolish containing from 20 to 55 g/L of ethanol, preferably from 35 to 50 g/L of ethanol and the application of a composition comprising a live leaven and/or a live yeast on the cooked bread.

According to an advantageous embodiment of the invention, the poolish is incorporated with all of the breadmaking ingredients in the kneading step.

According to another aspect, it is proposed to use a poolish containing from 20 to 55 g/L of ethanol, preferably from 35 to 50 g/L of ethanol, jointly with the application of a composition of live leaven and/or of live yeast on the surface of a cooked bakery product, to slow down the appearance of molds on the cooked bakery product, especially if sliced.

According to yet another aspect, the invention relates to cooked bakery products, preferably sliced, having a shelf life of at least 21 days, preferably of at least 28 days, which do not comprise non-natural additives.

### Disclosure of the Invention

The invention relates to a method for preparing mold-resistant cooked bakery products comprising cooking a dough obtained by a breadmaking method of the sponge and dough type using a poolish containing from 20 to 55 g/L of ethanol, preferably from 35 to 50 g/L of ethanol, and the application of a composition comprising a live leaven and/or a live yeast on the cooked bread.

This method is particularly useful for cooked bakery products incorporating sugar and fats, such as sandwich bread or American breads. In the majority of cases these cooked bakery products are offered to customers in the form of a packaged sliced product. It is important that they can be stored for several weeks.

In the sense of the present invention, “preservation” means absence of the development of molds on the surface of the cooked bakery product and maintenance of the organoleptic qualities of the cooked bakery product for at least 21 days, preferably at least 28 days at room temperature.

By employing an indirect method with a poolish combined with the application of a live leaven and/or of a live yeast after cooking, it becomes possible to increase the shelf life of the cooked bakery product while delaying the formation of molds.

The terms pre-ferment or poolish or sponge will be used without distinction hereinafter.

Even if the ethanol content in the poolish used in the method according to the invention may be high, it does not affect the organoleptic qualities of the cooked bakery product finally obtained. A large amount, or even most of the ethanol evaporates during cooking. The ethanol content after cooking is of the order of a few % (w/w), generally between 0.4 and 3% (w/w) and preferably between 0.5 and 1.5% (w/w).

According to one embodiment of the invention, the poolish is obtained by the maturation of a composition comprising flour, water, optionally sugar and/or enzymes by at least one yeast selected from the group comprising wine yeast, brewer's yeast and/or baker's yeast. According to the invention, “poolish containing from 20 to 55 g/L of ethanol, preferably from 35 to 50 g/L of ethanol” means a poolish that produces from 20 to 55 g/L of ethanol, preferably from 35 to 50 g/L of ethanol, during maturation of the composition comprising flour, water, optionally sugar and/or enzymes, by wine yeast, brewer's yeast and/or baker's yeast.

The wine yeast, brewer's yeast and/or baker's yeast used for maturation of the composition comprising flour, water, optionally sugar and/or enzymes, may be called “prefermentation yeast”. Preferably, this prefermentation yeast is a yeast obtained by culture of strains of Saccharomyces, preferably Saccharomyces cerevisiae. As examples, we may mention the commercial products with the following references: Safale S04, Saflager W34/70, Safale US-05, Safbrew F2, Saflager S-189, Safbrew T58, Safoeno BC S103, Safale BE-134, Safale BE-256, Safale K97, Safoeno CK S102, Safbrew WB06, Safale S23, Florapan Lallemand, UCLM S377, Saf Instant Red, Saf Instant Gold, Hirondelle Bleue compressed yeast, Hirondelle Or compressed yeast.

According to one embodiment of the invention, the duration of the poolish preparation step, i.e. the maturation time, may be similar to the conventional duration of the prefermentation step of a sponge and dough method, i.e. from 3 to 12 hours, but it may be longer. This duration is generally at least 4 hours, or at least 8 hours and even at least 12 hours. This duration may be up to 24 hours. As an example, we may mention a duration of 18 hours, and preferably of 16 hours. Still as an example, we might also mention a duration of 4 hours. However, if the time is too short (less than 3 hours), the preservation effect may not be optimal. However, for economic reasons, it is obvious that a duration exceeding 24 hours is not recommended.

According to another embodiment of the invention, the poolish/total flour ratio is adjusted so as not to alter the organoleptic properties of the bread.

In particular, the amount of flour in the poolish comprises from 20 to 65% of the total flour, preferably from 25 to 55%, more preferably 30% or 50% of the total flour. Below 20%, the antimold effect might prove insufficient.

The method of preparation according to the invention comprises a step of applying a composition comprising a live leaven and/or a live yeast on the surface of the cooked bakery product.

According to one embodiment, the composition applied on the surface of the cooked bakery product may comprise live leaven and live yeast.

According to yet another embodiment, the composition applied on the surface of the cooked bakery product may comprise live leaven or live yeast.

According to the invention, “composition comprising a live leaven” means any composition of liquid leaven, regardless of its manner of preparation.

According to one embodiment of the invention, the leaven used is a leaven based on baking flour, for example wheat flour, hard wheat flour, rye flour, white rye flour or buckwheat flour.

According to another embodiment of the invention, the composition of liquid live leaven is applied at a rate from 2 to 8 g, preferably from 3 to 7 g and even more preferably from 4 to 6 g of liquid leaven having dry matter from 15 to 20% on a total surface area of cooked bakery product from 1200 to 1900 cm².

In the composition of live yeast according to the invention applied on the surface of cooked bakery products, the yeast is the same as that used in the poolish or it is different. It is a wine yeast, brewer's yeast and/or baker's yeast.

Preferably, this yeast is obtained by culture of strains of Saccharomyces, preferably Saccharomyces cerevisiae. As examples, we may mention the commercial products with the following references: Safale S04, Saflager W34/70, Safale US-05, Safbrew F2, Saflager S-189, Safbrew T58, Safoeno BC S103, Safale BE-134, Safale BE-256, Safale K97, Safoeno CK S102, Safbrew WB06, Safale S23, Florapan Lallemand, UCLM S377, Saf Instant Red, Saf Instant Gold, Hirondelle Bleue compressed yeast, Hirondelle Or compressed yeast.

According to yet another embodiment of the invention, the composition of live liquid yeast is applied at a rate from 2 to 8 g, preferably from 3 to 7 g and even more preferably from 4 to 6 g of liquid composition having an amount of live yeast of the order of 10⁷ to 10¹⁰ CFU/L, preferably of the order of 10⁹ CFU/L on a total surface area of cooked bakery product from 1200 to 1900 cm².

According to yet another embodiment of the invention, the composition is applied on the whole surface of the cooked bakery product. In particular, in the case of a sliced cooked bakery product, the composition is advantageously applied on all the external surfaces of the slices.

The composition of live leaven and/or of liquid yeast may be applied by any known means such as spraying or dipping. The preferred means is spraying.

The method according to the invention is entirely suitable for the preparation of sourdough bread, sandwich bread, milkbread, milk loaf, brioche, buns or any other long-life product.

In particular, the cooked bakery products prepared according to the method of the invention are intended to be packaged in paper packaging or packaging of food-grade plastic film.

According to a particular embodiment, the method of the invention further comprises a step of packaging the cooked bakery product and a step of spraying the composition of live leaven and/or of live yeast in said packaging before it is closed.

According to another aspect, it is proposed to use a poolish containing from 20 to 55 g/L of ethanol, preferably from 35 to 50 g/L of ethanol, jointly with the application of a composition of live leaven and/or of live yeast on the surface of a cooked bakery product, to slow down the appearance of molds on cooked bakery products, in particular if sliced.

According to an advantageous embodiment of the invention, the flour of the poolish represents from 20 to 65 wt %, preferably at least from 25 to 55 wt % and in particular 30 wt % or 50 wt % of the total flour of the bread in which the poolish is present.

According to another embodiment, the poolish used is a poolish of at least 4 hours of maturation, preferably 8 hours of maturation, and even more preferably of at least 12 hours of maturation.

According to another embodiment, the composition of live leaven and/or of live yeast may additionally be sprayed in the closed packaging in which it is stored.

According to yet another aspect, the invention further relates to cooked bakery products, preferably sliced, having a shelf life of at least 21 days, preferably at least 28 days, and which do not comprise non-natural additives.

According to another embodiment of the invention, the cooked bakery products, preferably sliced, are selected from sourdough breads, sandwich loaves, milk loaves, milkbreads, brioches, buns or any other long-life product.

According to one embodiment of the invention, the cooked bakery products are sliced and packaged in closed packaging which is paper packaging or packaging of food-grade plastic film.

The cooked bakery products may be prepared by the method described above.

The invention will be described in more detail with the aid of the following examples, which are given purely for purposes of illustration, and the figures, where:

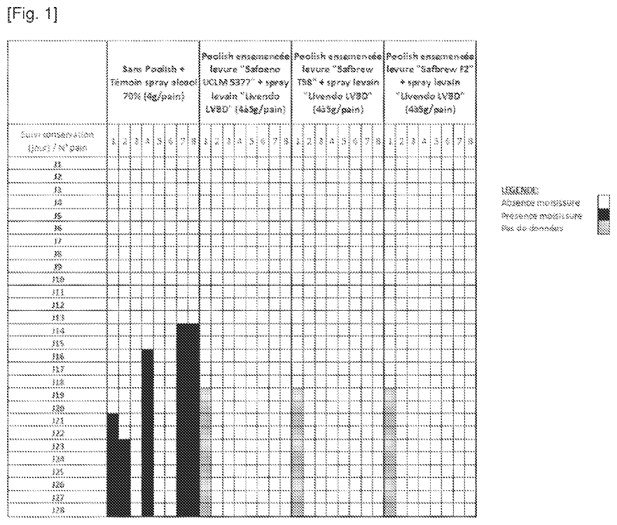

FIG. 1 is a chart showing the shelf life of loaves prepared according to example 1.

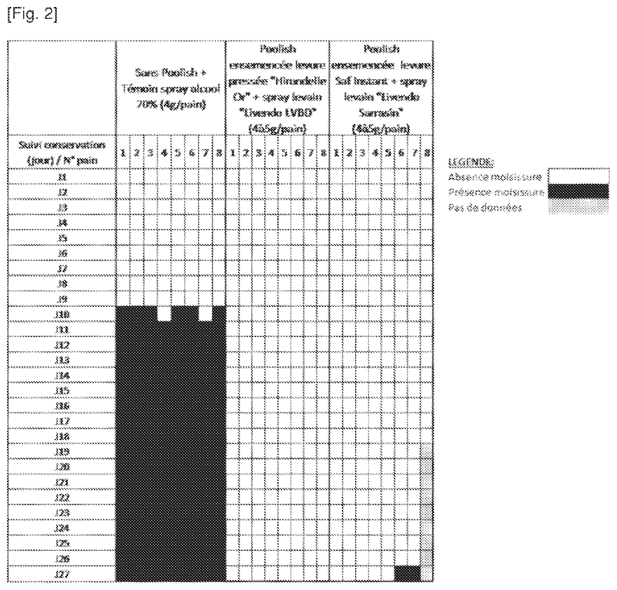

FIG. 2 is a chart showing the shelf life of loaves prepared according to example 2.

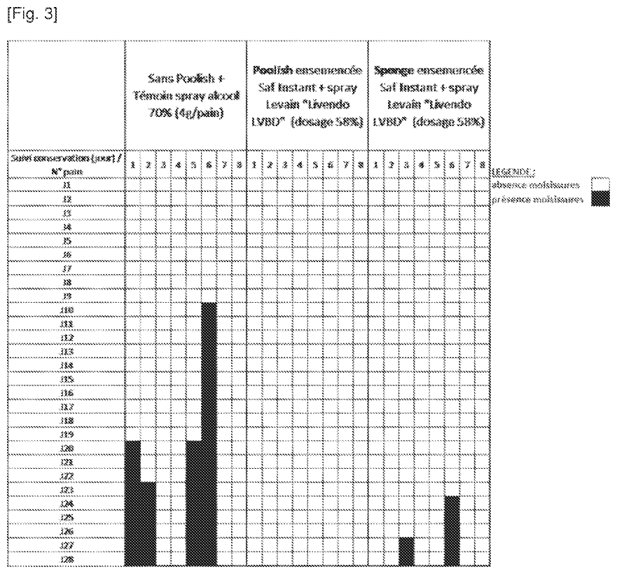

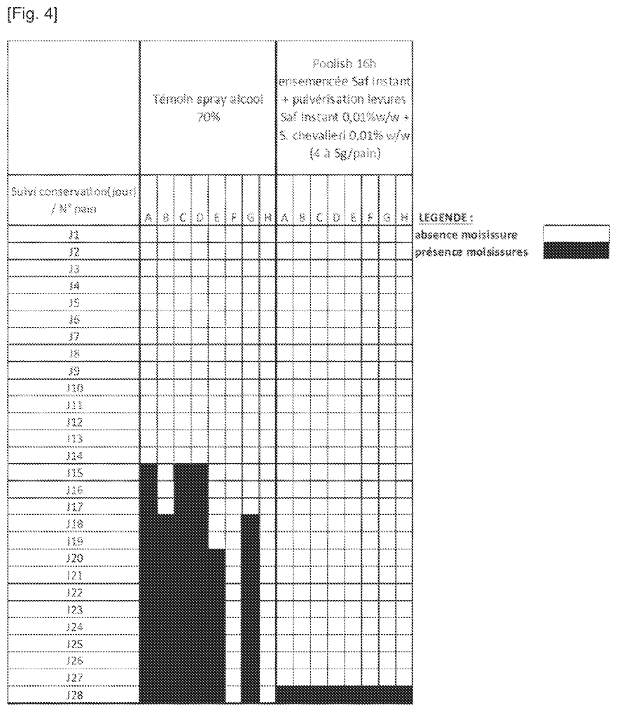

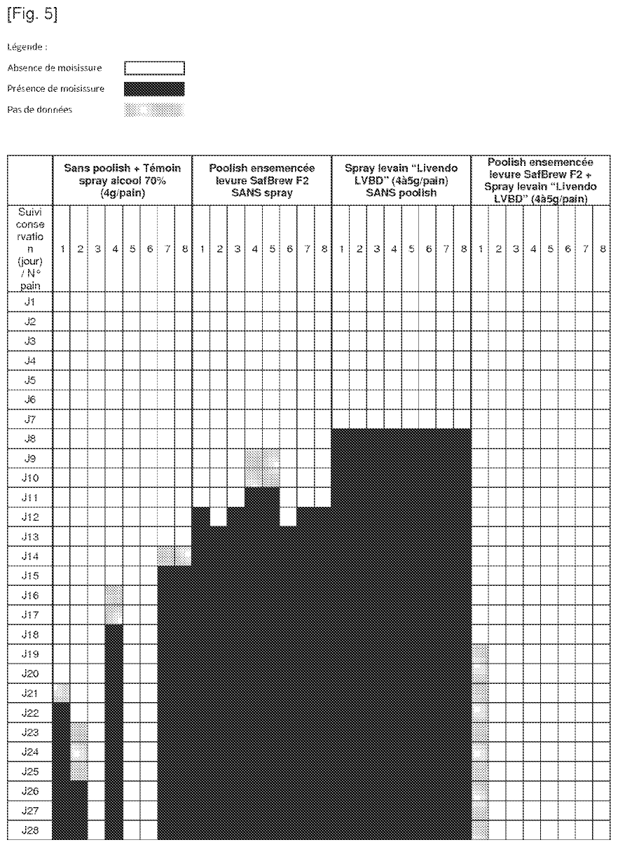

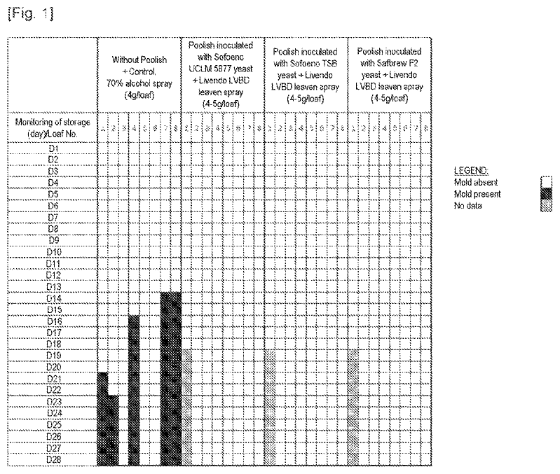

FIG. 3 is a chart showing the shelf life of loaves prepared according to example 3.

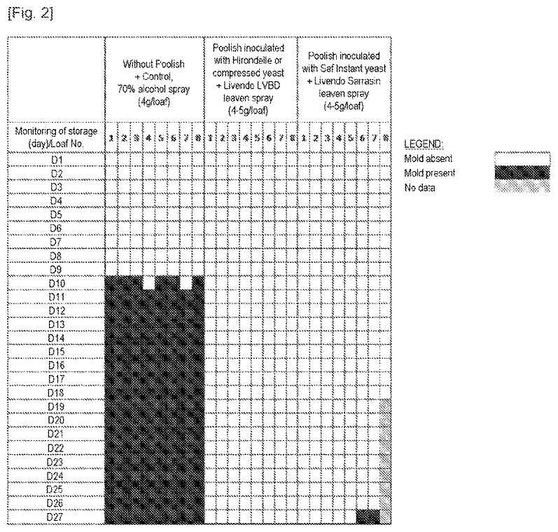

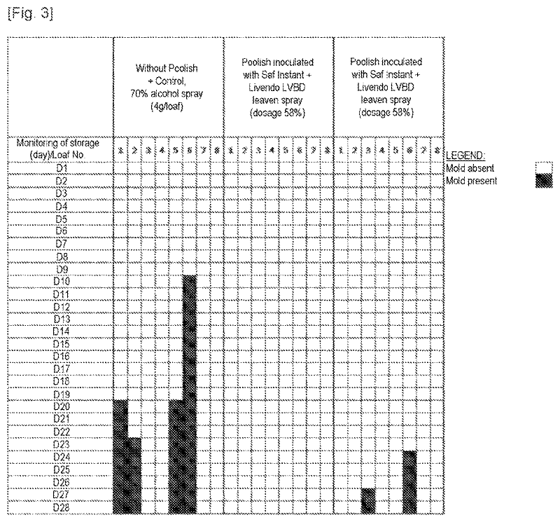

FIG. 4 is a chart showing the shelf life of loaves prepared according to example 4.

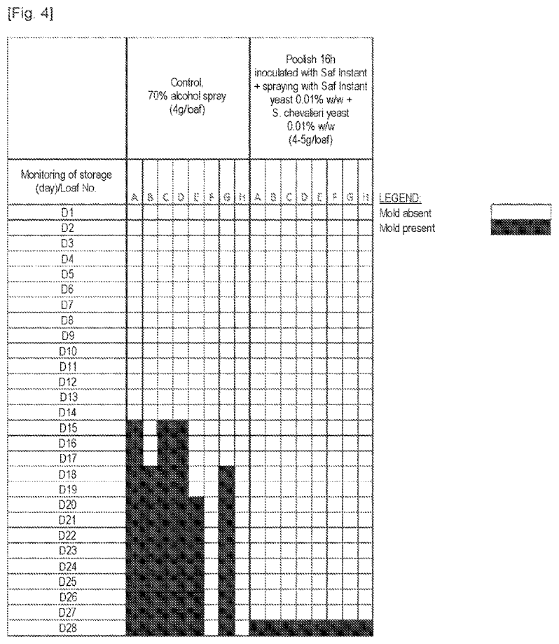

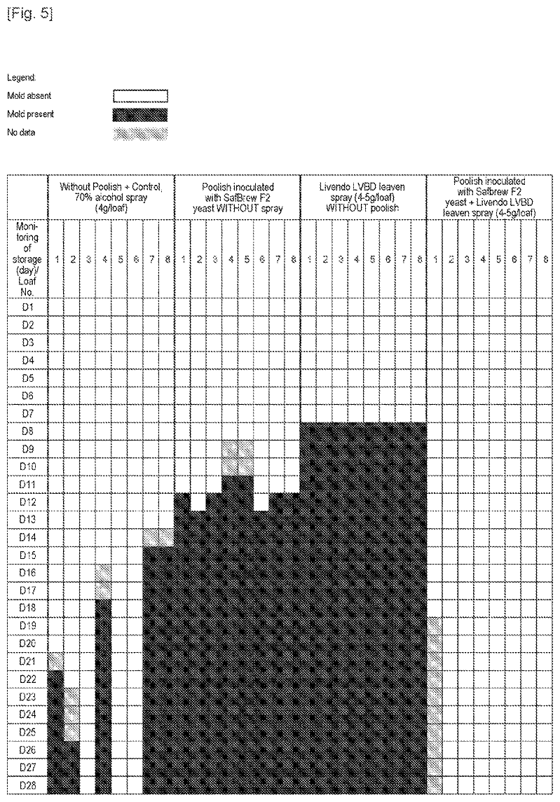

FIG. 5 is a chart showing the shelf life of loaves prepared according to example 5.

### EXAMPLES

### Example 1

The reference method used by the applicant is a sponge and dough method, where the sponge step and the dough step have the following characteristics:

This method was used for preparing 4 batches of sandwich bread. Each batch comprises 8 sandwich loaves. The first batch is a reference batch, without poolish, in which alcohol was sprayed on the cooked sandwich loaf at a rate of 4 g/cooked sandwich loaf. The other 3 batches are according to the invention and they each use a different yeast in the poolish, namely:
Batch 1: Yeast marketed by Lesaffre under the name Safoeno UCLM S377,
Batch 2: Yeast marketed by Lesaffre under the name Safbrew T58,
Batch 3: Yeast marketed by Lesaffre under the name Safbrew F2.

The cooked sandwich loaf obtained with the poolish of each of these batches is then sprayed with a Livendo® LVBD leaven composition.

“Hirondelle Bleue®” compressed yeast (denoted LP HB hereinafter) was used in the dough, for all the batches.

The improver Ibis Violet® is a breadmaking improver, marketed by Lesaffre, consisting of emulsifiers and giving a better volume of the loaf and better shelf life of the dough.

“Enz Pro 404” denotes a commercial mixture of alpha-amylase and amyloglucosidase.

The ingredients used at each step are as follows:

TABLE 1

| | | | Total |
|---|---|---|---|
| | Poolish | Dough | recipe |
| Traditional | 1100 | 2900 | 4000 |
| flour | | | |
| Water | 1100 | 1100 | 2200 |
| Yeast | 5.5 g yeast | 103.5 g | 5.5 g yeast + |
| | (Safoeno UCLM | LP HB | 103.5 g LP |
| | S377 or | | HB |
| | Safbrew T58 or | | |
| | Safbrew F2) | | |
| Salt | 0 | 80 | 80 |
| Ibis violet | 0 | 16 | 16 |
| improver | | | |
| Sugar | 110 | 200 | 310 |
| Butter | 0 | 200 | 200 |
| Enz Pro 404 | 11 | 0 | 11 |

The poolish are fermented for 16 h at 30° C.

Spraying Protocol

Spraying is carried out with the Dubor sprayer for the Livendo® LVBD leaven (TSA5 Dübor manual microspraying device).

Precautions for sterilization=sterilization of the grilles in the oven+cleaning the hands with alcohol before handling the loaves.

Spraying is carried out at a distance of between 20 and 30 cm from the product on all faces of the sandwich loaf. The intended total amount of solution of leaven sprayed is about 4 to 5 g per loaf.

The loaves, unsliced, are sprayed and packaged individually after sweating for 90 min (T=35° C. core temperature).

The loaves are stored in a cupboard, in the bakery, at room temperature.

### Monitoring Storage

Visual evaluation of the development of molds on the sandwich loaves. Daily recording of the number of loaves with/without mold.

The results are presented in FIG. 1.

The loaves are separated into 3 batches; each of the batches is treated, packaged and stored as in example 1. Formation of molds is observed with the naked eye every day.

The results are given in FIG. 1. It can be seen in FIG. 1 that the loaves with poolish, treated by spraying with leaven, have a longer shelf life than the loaves without poolish, treated by spraying with alcohol, regardless of the nature of the yeast used for the poolish.

### Example 2

The reference method used by the applicant is a sponge and dough method, where the sponge step and the dough step have the following characteristics:

This method was used for preparing 3 batches of sandwich bread. Each batch comprises 8 sandwich loaves. The first batch is a reference batch, without poolish, in which alcohol was sprayed on the cooked sandwich loaf at a rate of 4 g/cooked sandwich loaf. The other 2 batches are according to the invention and they each use different leavens during spraying on the cooked sandwich loaf:
Batch 1: Livendo® LVBD hard wheat leaven (+ yeast marketed by Lesaffre under the name Saf Instant in the poolish),
Batch 2: Livendo® LVSN buckwheat leaven (+ yeast marketed by Lesaffre under the name Saf Instant® in the poolish). “Hirondelle Bleue®” compressed yeast (denoted LP HB hereinafter) was used in the dough, for all the batches.

The ingredients used in each step are as follows:

TABLE 2

| | | | Total |
|---|---|---|---|
| | Poolish | Dough | recipe |
| Traditional | 1100 | 2900 | 4000 |
| flour | | | |
| Water | 1100 | 1100 | 2200 |
| Yeast | 5.5 g Saf | 103.5 g LP HB | 5.5 g Saf |
| | Instant yeast | | Instant |
| | | | yeast + |
| | | | 103.5 g LP |
| | | | HB |
| Salt | 0 | 80 | 80 |
| Ibis violet | 0 | 16 | 16 |
| improver | | | |
| Sugar | 110 | 200 | 310 |
| Butter | 0 | 200 | 200 |
| Enz Pro 404 | 11 | 0 | 11 |

The poolish are fermented for 16 h at 30° C.

### Spraying Protocol

Spraying carried out with the Dubor sprayer for the Livendo® LVBD leaven and for the Livendo® LVSN leaven (TSA5 Dübor manual microspraying device).

Precautions for sterilization=sterilization of the grilles in the oven+cleaning the hands with alcohol before handling the loaves.

Spraying is carried out at a distance of between 20 and 30 cm from the product on all faces of the sandwich loaf. The intended total amount of solution of leaven sprayed is about 4 to 5 g per loaf.

The loaves, unsliced, are sprayed and packaged individually after sweating for 90 min (T=35° C. core temperature).

The loaves are stored in a cupboard, in the bakery, at room temperature.

Monitoring Storage

Visual evaluation of the development of molds on the sandwich loaves. Daily recording of the number of loaves with/without mold.

The results of the visual evaluation are given in FIG. 2. It can be seen in FIG. 2 that the loaves with poolish, treated by spraying of leaven, have a longer shelf life than the loaves without poolish, treated by spraying with alcohol, regardless of the live leaven used for spraying.

### Example 3

This method was used for preparing 3 batches of sandwich bread. Each batch comprises 8 sandwich loaves. The first batch is a reference batch without poolish, in which alcohol was sprayed on the cooked sandwich loaf at a rate of 4 g/sandwich loaf. The other 2 batches are according to the invention and use of prefermentations with various water contents (the leaven yeast prepared in the prefermentation step will be called poolish or sponge, depending on the water content):

### Recipe & Process for Preparing the Poolish:

### Recipe:

100% traditional wheat flour,
100% water (32° C.),
1% Enz Pro 404,
0.5% SAF Instant® yeast,
10% glucose.

### Process:

24 h of maturation,
30° C.,
No stirring.
   Recipe & process for preparing the sponge:

### Recipe:

100% traditional wheat flour,
50% water (32° C.),
1% Enz Pro 40,
0.5% SAF Instant®,
10% glucose.

### Process:

24 h of maturation,
30° C.,
No stirring.

Table 3 below gives the recipe and process for the sandwich bread without poolish (control).

TABLE 3

| Ingredients | weight | % |
|---|---|---|
| Traditional | 4000 | 100 |
| flour | | |
| Water | 2200 | 55 |
| LP HB yeast | 103.5 | 3.0 |
| Salt | 80 | 1.8 |
| Ibis violet | 16 | 0.5 |
| improver | | |
| Sugar | 200 | 5.0 |
| Butter | 200 | 5.0 |

TABLE 4
Breadmaking chart

| STEPS | DOUGH | |
|---|---|---|
| kneading | 4 + 6.5 | min |
| initial fermentation | 5 | min |
| Division I | 720 | g |
| rounding | yes | |
| expanding | 10 | min |

| | |
|---|---|
| Shaping | Machine/twisted |
| Final proving 1 | 30° C./85% |
| 80 grams of dough: 260 ml | moisture, 80 min |
| Cooking | 25 min 220° C. |

Table 5 below presents the recipe and process for the sandwich bread with poolish or sponge.

TABLE 5

| | Poolish or | | Total |
|---|---|---|---|
| Ingredients | sponge | Dough | recipe |
| Traditional | 1100 | 2900 | 4000 |
| flour | | | |
| Water | 1100 | 1100 | 2200 |
| | (poolish) | | |
| | or 550 | | |
| | (sponge) | | |
| Yeast | 5.5 g Saf | 103.5 LP HB | 5.5 g Saf |
| | Instant yeast | | Instant |
| | | | yeast + |
| | | | 103.5 g LP |
| | | | HB |
| Salt | 0 | 80 | 80 |
| Ibis violet | 0 | 16 | 16 |
| improver | | | |
| Sugar | 110 | 200 | 310 |
| Butter | 0 | 200 | 200 |
| Enzymes Pro | 11 | 0 | 11 |
| 404 | | | |

Spraying Protocol

Manual spraying carried out with manual sprayers for the Livendo LVBD leavens.

Precautions for sterilization=sterilization of the grilles in the oven+cleaning the hands with alcohol before handling the loaves.

Spraying is carried out at a distance of between 20 and 30 cm from the product on all faces of the sandwich loaf. The LVBD leaven was centrifuged beforehand to prevent clogging of the sprayer nozzle.

The intended total amount of solution of leaven sprayed is about 4 to 5 g with the garden sprayer.

The loaves, unsliced, are sprayed and packaged individually after sweating for 90 min (T=35° C. core temperature).

The loaves are stored in a cupboard, in the bakery, at room temperature.

Monitoring Storage

Visual evaluation of the development of molds on the sandwich loaves. Daily recording of the number of loaves with/without mold.

Note: industrial sandwich loaves conventionally have a best before date (BBD) of 21-28 days.

Visual observation was performed and the results are presented in FIG. 3. It can be seen in FIG. 3 that the loaves with poolish or sponge, treated by spraying with leaven, have a longer shelf life than the loaves without poolish, treated by spraying with alcohol, regardless of the water content in prefermentation (poolish or sponge).

### Example 4

The reference method used by the applicant is a sponge and dough method, where the sponge step and the dough step have the following characteristics:

This method was used for preparing 2 batches of sandwich bread. Each batch comprises 8 sandwich loaves. The first batch is a reference batch, without poolish, in which alcohol was sprayed on the cooked sandwich loaf at a rate of 4 g/cooked sandwich loaf. The other batch is according to the invention and uses incorporation of a poolish inoculated with Saf Instant yeast and spraying the cooked product with a solution of live yeasts (mixture of Saf Instant yeasts and Saccharomyces chevalieri each at a dose of 0.01% w/w and diluted in water at 30° C.).

“Hirondelle Bleue®” compressed yeast (denoted LP HB hereinafter) was used in the dough, for all the batches.

The ingredients used in each step are as follows:

TABLE 6

| | | | Total |
|---|---|---|---|
| | Poolish | Dough | recipe |
| Traditional | 1100 | 2900 | 4000 |
| flour | | | |
| Water | 1100 | 1100 | 2200 |
| Yeast | 5.5 g Saf | 103.5 LP HB | 5.5 g Saf |
| | Instant yeast | | Instant |
| | | | yeast + |
| | | | 103.5 g LP |
| | | | HB |
| Salt | 0 | 80 | 80 |
| Ibis violet | 0 | 16 | 16 |
| improver | | | |
| Sugar | 110 | 200 | 310 |
| Butter | 0 | 200 | 200 |
| Enzymes | 11 | 0 | 11 |

The poolish are fermented for 16 h at 30° C.

Spraying Protocol

Spraying carried out with the Dubor sprayer for the solution of live yeasts (TSA5 Dübor manual microspraying device).

Precautions for sterilization=sterilization of the grilles in the oven+cleaning the hands with alcohol before handling the loaves.

Spraying is carried out at a distance of between 20 and 30 cm from the product on all faces of the sandwich loaf. The intended total amount of solution of yeasts sprayed is about 4 to 5 g per loaf.

The loaves, unsliced, are sprayed and packaged individually after sweating for 90 min (T=35° C. core temperature).

The loaves are stored in a cupboard, in the bakery, at room temperature.

Monitoring Storage

Visual evaluation of the development of molds on the sandwich loaves. Daily recording of the number of loaves with/without mold.

The results are presented in FIG. 4.

Formation of molds is observed with the naked eye every day.

The results are given in FIG. 4.

It can be seen in FIG. 4 that the loaves with poolish, treated by spraying with a solution of yeasts, have a longer shelf life than the loaves without poolish, treated by spraying with alcohol.

### Example 5

This example is an illustration of the synergistic effect between poolish and spraying the surface of the cooked bakery product with a solution comprising a live leaven.

The reference method used by the applicant is a sponge and dough method, where the sponge step and the dough step have the following characteristics:

This method was used for preparing 4 batches of sandwich bread. Each batch comprises 8 sandwich loaves. The first batch is a reference batch, without poolish, in which alcohol was sprayed on the cooked sandwich loaf at a rate of 4 g/cooked sandwich loaf. In the second batch, the sandwich loaves are made by the sponge and dough method starting from yeast marketed by Lesaffre under the name Safbrew F2 but without spraying after cooking. In the third batch, the sandwich loaves do not contain poolish, but are sprayed with a Livendo® LVBD leaven composition after cooking. The fourth batch is according to the invention; the poolish is made starting from yeast marketed by Lesaffre under the name Safbrew F2 and the cooked sandwich bread obtained with poolish is then sprayed with a Livendo® LVBD leaven composition.

“Hirondelle Bleue®” compressed yeast (denoted LP HB hereinafter) was used in the dough, for all the batches.

The ingredients used in each step are as follows:

TABLE 7

| | | | Total |
|---|---|---|---|
| | Poolish | Dough | recipe |
| Traditional | 1100 | 2900 | 4000 |
| flour | | | |
| Water | 1100 | 1100 | 2200 |
| Yeast | 5.5 g Safbrew | 103.5 LP HB | 5.5 g yeast + |
| | yeast F2 | | 103.5 g LP |
| | | | HB |
| Salt | 0 | 80 | 80 |
| Ibis violet | 0 | 16 | 16 |
| improver | | | |
| Sugar | 110 | 200 | 310 |
| Butter | 0 | 200 | 200 |
| Enzymes | 11 | 0 | 11 |

The poolish are fermented for 24 h at 30° C.

TABLE 8

| | Dough |
|---|---|
| Traditional flour | 4000 |
| Water | 2200 |
| Yeast | 103.5 g LP HB |
| Salt | 80 |
| Ibis violet improver | 16 |
| Sugar | 200 |
| Butter | 200 |
| Enz Pro 404 | 11 |

### Spraying Protocol

Spraying is carried out with the Dubor sprayer for Livendo® LVBD leaven (TSA5 Dübor manual microspraying device).

Precautions for sterilization=sterilization of the grilles in the oven+cleaning the hands with alcohol before handling the loaves.

Spraying is carried out at a distance of between 20 and 30 cm from the product on all faces of the sandwich loaf.

The intended total amount of solution of leaven sprayed is about 4 to 5 g per loaf.

The loaves, unsliced, are sprayed and packaged individually after sweating for 90 min (T=35° C. core temperature).

The loaves are stored in a cupboard, in the bakery, at room temperature.

Monitoring Storage

Visual evaluation of the development of molds on the sandwich loaves. Formation of molds is observed with the naked eye every day.

Daily recording of the number of loaves with/without mold.

Note: industrial sandwich loaves conventionally have a BBD of 21-28 days.

The results of the visual evaluation are given in FIG. 5. It can be seen in FIG. 5 that the sandwich loaves with poolish and without spraying with leaven and the sandwich loaves without poolish but sprayed with leaven suffer earlier appearance of molds than the sandwich loaves with poolish and sprayed with leaven. This is evidence of a synergistic effect between poolish and spraying with leaven. It is the combined action of the two elements (poolish+spraying) that makes it possible to obtain an increased antimold effect using only natural products.

1.-17. (canceled)

18. A method for preparing mold-resistant cooked bakery products comprising cooking a dough obtained by a breadmaking method of the sponge and dough type using a poolish containing from 20 to 55 g/L of ethanol, preferably from 35 to 50 g/L of ethanol, and comprising, after cooking, a step of application of a solution comprising a live leaven and/or a live yeast on the surface of the cooked bakery product.

19. The method as claimed in claim 18, wherein said poolish is obtained by the maturation of a composition comprising flour, water, optionally sugar and/or enzymes by at least one yeast selected from the group comprising wine yeast, brewer's yeast and/or baker's yeast.

20. The method as claimed in claim 18, wherein yeast is obtained by culture of the strain Saccharomyces cerevisiae.

21. The method as claimed in claim 18, wherein the maturation time of the poolish is at least 4 hours, preferably at least 8 hours and even more preferably at least 12 hours.

22. The method as claimed in claim 18, wherein the flour of the poolish represents 20 to 65% of the total flour, preferably 25 to 55%, even more preferably 30% or 50% of the total flour.

23. The method as claimed in claim 18, wherein the live leaven applied on the surface of the cooked bakery product is a leaven for any baking flour, preferably for wheat flour, hard wheat flour, rye flour, white rye flour or buckwheat flour.

24. The method as claimed in claim 18, wherein the composition of live leaven is applied at a rate from 2 to 8 g, preferably from 3 to 7 g and even more preferably from 4 to 6 g of liquid leaven having dry matter from 15 to 20% on a surface area of cooked bakery product from 1200 to 1900 cm².

25. The method as claimed in claim 18, wherein the composition of live yeast is applied at a rate from 2 to 8 g, preferably from 3 to 7 g and even more preferably from 4 to 6 g of liquid composition having a quantity of live yeasts of the order of 10⁷ to 10¹⁰ CFU/L, preferably of the order of 10⁹ CFU/L on a surface area of cooked bakery product from 1200 to 1900 cm².

26. The method as claimed in claim 18, wherein the composition of live leaven is applied by dipping or spraying.

27. The method as claimed in claim 18, for preparing sourdough bread, sandwich bread, milkbread, milk loaf, brioche, buns or any other long-life product.

28. The method as claimed in claim 18, The method as claimed in one of the preceding claims for preparing cooked bakery products, in particular sliced, intended to be packaged in paper packaging or in packaging made of food-grade plastic film.

29. A method of slowing down the appearance of molds on cooked bakery products, in particular if sliced, comprising including in a cooked bakery product a poolish containing from 20 to 55 g/L of ethanol, preferably from 35 to 50 g/L of ethanol, jointly with application of a composition of live leaven and/or of live yeast on the surface of the cooked bakery product.

30. The method as claimed in claim 29, wherein the flour of the poolish represents 20 to 65%, preferably 25 to 55% and in particular 30% or 50 wt % of the total flour of the bread in which the poolish is present.

31. The method as claimed in claim 29, wherein the composition of live leaven and/or of live yeast may additionally be sprayed in the closed packaging in which it is stored.

32. A cooked bakery product prepared as claimed in claim 18, preferably sliced, having a shelf life of at least 21 days, preferably of at least 28 days, and which does not comprise non-natural additives.

33. The cooked bakery product as claimed in claim 32, selected from sourdough bread, sandwich bread, milkbread, milk loaf, brioche, buns or any other long-life product.

34. The cooked bakery product as claimed in claim 32, which is a sliced loaf packaged in closed packaging selected from paper packaging or packaging made of food-grade plastic film.
